(19) United States
(12) Patent Application Publication
(10) Pub. No.: US 2017/0020810 A1
(43) Pub. Date: Jan. 26, 2017

(54) Nutritional Supplement and Method of Administering the Same

(71) Applicant: Brian Street, Boca Raton, FL (US)

(72) Inventor: Brian Street, Boca Raton, FL (US)

(73) Assignee: NRG Innovations LLC, Deerfield Beach, FL (US)

(21) Appl. No.: 14/808,038

(22) Filed: Jul. 24, 2015

(51) Int. Cl. A61K 9/00; A23G 1/32; A61K 31/59; A61K 31/455; A61K 31/522; A61K 31/198; A61K 31/519; A23G 1/54; A61K 9/48

(52) U.S. Cl. CPC A61K 9/0056; A23L 1/303; A23G 1/54; A23G 1/32; A23L 1/30; A23L 1/302; A61K 31/59; A61K 9/4808; A61K 9/4816; A61K 31/522; A61K 31/198; A61K 31/519; A61K 31/455; A23V 2002/00

(57) ABSTRACT

A nutritional supplement that improves energy levels in a human being and a method of administering the same to provide enhanced energy is described. The nutritional supplement comprises fat, caffeine, vitamins and minerals enveloped and surrounded in an edible protective coating. In preferred embodiments, the nutritional supplement is a composition including a fatty matrix supporting caffeine, B vitamins and vitamin D, the fatty matrix is comprised of dark chocolate and the edible protective coating is a sugar shell. The caffeine in the supplement provides for enhanced energy, vitamin D helps to maintain muscle function and strength and supports mental health, and B vitamins facilitate conversion of food or stored energy into active energy. The fatty matrix and edible protective coating cooperate to preserve the nutritional supplement without the need for artificial preservatives. The fatty matrix and edible protective coating further provide for delivery and improved bioabsorption of the caffeine and vitamins to a human ingesting the food product. The composition also may include L-Theanine to assist in tempering the anxiety-inducing effects of caffeine such as jitteriness and masking agents to mask the bitterness of the caffeine, vitamins and minerals. The method comprises administration of the nutritional supplement as described to improve energy levels in a human being.

### TECHNICAL FIELD

The invention relates to a nutritional supplement (which can also be referred to as a “dietary supplement”) that can improve energy levels in a human being and a method of administering such a nutritional supplement.

### BACKGROUND

A variety of nutritional supplements and energy supplements in particular are available in the market. Most energy supplements include caffeine or an ingredient containing caffeine as an active ingredient. Energy supplements containing caffeine tend to promote a jittery or a nervous type of energy. Energy boosting drinks and gels containing caffeine have a reasonably long shelf life if left unopened. However, once a drink or gel containing caffeine is opened, the caffeine in the drink or gel will dissipate much more quickly and that opened product has a very limited shelf life.

Thus, there is a need in the art for a nutritional supplement that provides for increased alertness, focus and concentration without anxiety-inducing effects. Further, there is a need in the art for a nutritional supplement that provides for effective conversion of food and stored fuel to energy. Finally, there is a need in the art for a nutritional supplement having an extended shelf life without the need for artificial preservatives and artificial colors. These and other needs in the art are addressed in the nutritional supplemental and method of administering the same as described and claimed herein.

### SUMMARY

The invention relates to a nutritional supplement that can improve energy levels in a human being. The nutritional supplement comprises sources of fat, caffeine, vitamins and minerals enveloped in an edible protective coating. In preferred embodiments, the nutritional supplement is a composition including a fatty matrix supporting caffeine and vitamins B and D. More preferably, the fatty matrix is comprised of dark or white chocolate or caramel or peanut butter and the edible protective coating enveloping the fatty matrix is a sugar shell.

In the preferred embodiments, caffeine provides for enhanced energy, vitamin D helps to maintain muscle function and strength and supports mental health, and B vitamins facilitate conversion of food or stored energy into active energy. The fatty matrix and edible protective coating cooperate to preserve the nutritional supplement and, in particular, the caffeine contained in the nutritional supplement, without the need for artificial preservatives. The fatty matrix and edible protective coating further provide for constant delivery and improved bioabsorption of the caffeine, vitamins and minerals to a human ingesting the supplement. The composition also may include L-Theanine to assist in tempering the anxiety-inducing effects of caffeine and to assist with focus/attentional processing and a masking agent to mask the bitterness of the caffeine, vitamins and minerals.

The nutritional supplement can be made in a variety of forms, e.g. capsules, gems, or non-baked extruded food products. The preferred form of the nutritional supplement is a non-baked extruded food product with a center including a dark chocolate, white chocolate, caramel or peanut butter mass containing the caffeine, vitamins and minerals. The preferred form of the edible protective coating is a sugar shell enveloping and protecting the dark chocolate mass, which can be naturally colorful without the need for artificial colors. The supplement can be formulated for single or multiple daily administration, and is preferably formulated for administration three times a day.

The invention further pertains to therapeutic methods for enhancing energy levels, maintaining muscle function and strength, supporting mental health, facilitating conversion of food or stored energy into active energy, tempering the anxiety-inducing effects attendant to some energy supplements and generally supporting the mood and focus/attentional processing of the human subject. The method preferably comprises administration of a nutritional supplement having a composition including caffeine and vitamin D in an amount sufficient to improve energy levels in the human being and vitamin B in an amount sufficient to facilitate energy conversion, which are supported in a fatty matrix to provide for constant delivery to and improved bioabsorption of the caffeine, vitamins and minerals by the human being, and enveloped in an edible protective covering to maintain the efficacy of the caffeine, vitamins and minerals. The method comprises administering in the composition L-Theanine to temper anxiety-inducing effects of caffeine, improve focus/attentional processing and a masking agent to mask the bitterness of the caffeine, vitamins and minerals.

For a more complete explanation of the above and other features and advantages of the nutritional supplement, reference should be made to the following description of the preferred embodiments.

### BRIEF DESCRIPTION OF THE DRAWINGS

FIG. 1 is perspective view of a nutritional supplement.

FIG. 2 is a cross-section of the nutritional supplement at sectional lines 2-2 in FIG. 1.

### DETAILED DESCRIPTION OF PREFERRED EMBODIMENTS

The invention relates to a nutritional supplement containing active ingredients to boost energy in an individual. Clinically proven food grade nutrients are assembled in the dosages described to enhance energy levels. The supplement is composed in a manner that preserves the efficacy of the nutrients without the need for artificial preservatives.

More specifically, the nutritional supplement comprises fat, caffeine, vitamins and minerals. Caffeine in appropriate dosages increases alertness, focus and concentration. Vitamin D helps to maintain muscle function and strength and supports mental health. B vitamins facilitate conversion of food and stored fuel to energy. Other ingredients, such as vitamins A, C and E, herbs or minerals, may be added to the supplement to improve its support for these functions. For example, L-Theanine may be added to provide a calming effect and to temper the potential for jittery/anxiety-inducing effects of caffeine without compromising its beneficial effects, and can assist with focus/attentional processing. The caffeine, vitamins and any other active ingredients are dispersed in a fatty matrix which facilitates and improves bioadsorption of the active ingredients in the individual and serves to preserve the active ingredients. Further, the fat matrix is enveloped in an edible protective coating which further serves to preserve the active ingredients. The combination of supporting the active ingredients in a fatty matrix and enveloping the matrix in a protective coating results in a nutritional supplement with enhanced shelf life without the need for artificial preservatives.

In preferred embodiments, the nutritional supplement comprises approximately 50-250 mg of caffeine, 150-1000 IU of vitamin D, 5-25 mg of vitamin B3, 5-25 mg of vitamin B6, 25-150 mcg of B9, 50-400 mcg of vitamin B12 and 40-250 mg of L-Theanine in a fatty matrix. The ranges used herein are based on a single serving containing up to three capsules or gems, which are preferably formulated for administration daily in three parts over the course of a day. Additional vitamins and minerals, in amounts recommended daily to supplement the human diet, can also optionally be added.

The nutritional supplement can be made in a variety of forms, e.g. capsules or non-baked extruded food products. One preferred form of the nutritional supplement is a non-baked extruded food product including a dark or white chocolate mass containing the caffeine, vitamins and minerals. Other preferred embodiments include a non-baked extruded food products not containing chocolate such as a peanut butter or salted caramel mass. The preferred fon of the edible protective coating is a sugar shell enveloping the dark chocolate mass. The supplement can be formulated for single or multiple daily administration, and is preferably formulated for administration up to three times a day.

The nutritional supplement can be formulated as a snack to be taken as supplemental nutrition for the diet, or can be formulated as meal replacement. As a meal replacement, the supplement should provide about 200-400 kcal per serving and should include protein in addition to carbohydrates, fats, and caffeine.

Caffeine

Caffeine is a bitter, white crystalline xanthine alkaloid and psychoactive stimulant that is soluble in water. Caffeine is found in varying quantities in the seeds, leaves, and fruit of some plants. It is most commonly consumed by humans in infusions extracted from the bean of the coffee plant and the leaves of the tea bush. Other sources include yerba mate, guarana berries, guayusa, and the yaupon holly. Caffeine acts as a central nervous system stimulant, temporarily warding off drowsiness and increasing alertness. Caffeine has been shown to improve long-term cognitive performance, mood, and alertness in well-rested and sleep-deprived individuals without side effects. DAVID M. PENETAR ET AL., Effects of Caffeine on Cognitive Performance, Mood, and Alertness in Sleep-Deprived Humans, in FOOD COMPONENTS TO ENHANCE PERFORMANCE 407-431 (1994); PAULA J. DURLACH, The Effect of Low Dose of Caffeine on Cognitive Performance, in PSYCHOPHARMACOLOGY 116-119 (1998); PARSONS NEIMS & R. W. VON BORSTEL, Caffeine: Metabolism and Biochemical Mechanisms of Action, in NUTRITION AND THE BRAIN 1-30 (R J Wurtman ed., 1983). In the body, caffeine operates on receptors for adenosine—a naturally occurring brain chemical which signals fatigue. Adenosine is released into the blood with prolonged lack of sleep and also as available daylight dims. There is an important difference between this signal of fatigue and actual fatigue. Using caffeine does not eliminate the need for sleep, just puts the signal for sleep on hold for a while.

In addition to the anti-fatigue qualities of caffeine, there is also evidence that caffeine can increase athletic performance in endurance sports, possibly by enhancing metabolic activation of stored energy. MICHAEL J. GLADE, CAFFEINE—NOT JUST A STIMULANT (2010), available at PubMed 20888549.

The precise amount of caffeine necessary to produce effects varies from person to person depending on body size and degree of tolerance to effects of caffeine. Small amounts of caffeine (50 mg) found in a cup of tea or ⅓ of a cup of strong coffee appear to be effective in most people. CORINNE B. CATALDO & ELEANOR N. WHITNEY, NUTRITION & DIET THERAPY: PRINCIPLES AND PRACTICE 64 (4th ed. 1995). Doses of up to 500 mg per day are deemed safe and effective. Indications differ for pregnant women and children. It normally takes less than an hour for caffeine to begin affecting the body. Caffeine has its maximum effect within 2 to 4.5 hours of ingestion and then is almost entirely excreted.

Nutritional supplements designed to improve energy and alertness typically contain caffeine or other ingredients (e.g. green tea, guarana) containing caffeine. Many energy supplements contain 200-250 mg of caffeine per serving.

The amount of caffeine incorporated into the nutritional supplement should be that which provides from about 40 mg to about 400 mg caffeine, with from about 50 mg to about 250 mg being preferred. In the most preferred embodiment, the amount of caffeine in the nutritional supplement will be about 100 mg per serving, or the equivalent of one strong or large cup of coffee. The nutritional supplement is preferably administered in a serving of up to three parts during the course of a day including approximately 35 mg of caffeine per individual capsule or gem.

Fats

The nutritional supplement includes from about 1 to about 50 g fat, with from about 2 g to about 10 g being preferred and from about 2.5 g being most preferred. Sources of fats can include but are not limited to cocoa butter and oils present in the fatty matrix of dark chocolate.

B Vitamins

Generally, the B vitamins serve as enzyme enablers (co-enzymes) necessary to convert food or stored energy in the form of lipids, carbohydrates and proteins into active energy in the form of ATP (adenosine triphosphate). DIETARY REFERENCE INTAKES FOR THIAMIN, RIBOFLAVIN, NIACIN, VITAMIN B6, FOLATE, VITAMIN B12, PANTOTHENIC ACID, BIOTIN, AND CHOLINE 150 (The National Academies Press 1998).

Vitamin B1, also known as thiamin, helps convert blood sugar into energy. Thiamin is essential for the metabolism of carbohydrates to produce energy and for normal nerve and heart function.

Riboflavin, vitamin B2, is important for body growth and red blood cell production and helps in releasing energy from carbohydrates. Riboflavin, as flavin mononucleotide or flavin adenine dinucleotide, acts as an essential coenzyme in many oxidation-reduction reactions involved with carbohydrate metabolism.

Niacin, vitamin B3, is required for cell respiration, helps in the release of energy and metabolism of carbohydrates, fats, and proteins, proper circulation and healthy skin and functioning of the nervous system.

Vitamin B6 is needed for enzymes involved in protein metabolism. It is also essential for red blood cell metabolism. The body needs vitamin B6 to make hemoglobin which carries oxygen to tissues. Vitamin B6 also helps increase the amount of oxygen carried by hemoglobin. A vitamin B6 deficiency can result in a form of anemia that is similar to iron deficiency anemia. Vitamin B6 also helps maintain blood glucose (sugar) within a normal range. When caloric intake is low, the body needs vitamin B6 to help convert stored carbohydrate or other nutrients to glucose to maintain normal blood sugar levels.

Pantothenic acid, vitamin B5, participates in the continual breakdown of carbohydrates, fats, and proteins from food, converting them into compounds the body can use.

Folic acid, vitamin B9, works with vitamin B12 and vitamin C to help the body digest and utilize proteins and to synthesize new proteins when they are needed. It is necessary for the production of red blood cells and for the synthesis of DNA. Folic acid also helps with tissue growth and cell function.

Vitamin B12 helps maintain healthy nerve cells and red blood cells. It is also needed to help make DNA, the genetic material in all cells.

Many nutritional supplements contain B vitamins and vitamins B6 and B12 in particular.

Vitamin D

Vitamin D helps maintain muscle function and strength. Many people do not get enough Vitamin D from food or sunlight, so a majority of adults are low or deficient in vitamin D. Previously it was believed that vitamin D promoted absorption of calcium, but it is now clear that hundreds of functions throughout the body require vitamin D. ARASH HOSSEIN-NEZHAD & MICHAEL F. HOLICK, VITAMIN D FOR HEALTH: A GLOBAL PERSPECTIVE 720-755 (2013) available at http://www.mayoclinicproceedings.org/article/S0025-6196(13)00404-7/abstract. Two recent reviews confirm that low vitamin D is associated with loss of muscle strength; both of those reviews cite evidence that improving vitamin D status reduces the risk of falls that lead to fractures. PETER B. TOMLINSON ET AL., Effects of Vitamin D Supplementation on Upper and Lower Body Muscle Strength Levels in Health Individuals: A Systematic Review with Meta-Analysis, in JOURNAL OF SCIENCE AND MEDICINE IN SPORT 163-167 (2014); CRISTIANA CIPRIANI ET AL., Vitamin D and its Relationship With Obesity and Muscle, in INTERNATIONAL JOURNAL OF ENDOCRINOLOGY 1-11 (2014).

Vitamin D also supports mental health. Three systemic reviews and/or meta-analyses concluded that in epidemiological studies, lower vitamin D status was associated with poorer cognitive function and a higher risk of Alzheimer's disease and also a higher risk of depression. CEDRIC ANNWEILER ET AL., Meta-analysis of Memory and Executive Dysfunctions in Relation to Vitamin D, in JOURNAL OF ALZHEIMER'S DISEASE 147-171 (2013); CYNTHIA BALION ET AL., VITAMIN D, COGNITION AND DEMENTIA A SYSTEMATIC REVIEW AND META-ANALYSIS 1397-1405 (2012) available at PubMed 23008220; SANG-YHUN JU ET AL., Serum 25—Hydroxyvitamin D Levels and the Risk of Depression: A Systematic Review and Meta-analysis, in THE JOURNAL OF NUTRITION, HEALTH AND AGING 447-455 (2012).

The nutritional supplement preferably contains vitamin D3. The bioequivalence of the different forms of vitamin D, ergocalciferol (vitamin D2) and cholecalciferol (vitamin D3), has been questioned. Studies have suggested that vitamin D2 is less biologically active than vitamin D3. Results from an eight week clinical trials showed that Vitamin D3 increased total 25(OH)D concentration more than vitamin D2, supporting a claim that vitamin D3 is more effective. ULRIKE LEHMANN ET AL., Bioavailability of Vitamin D2 and D3 in Healthy Volunteers, a Randomized Placebo-Controlled Trial, in THE JOURNAL OF CLINICAL ENDOCRINOLOGY AND METABOLISM 4339-45 (2013). Two meta-analyses of clinical trials concluded that vitamin D3 lowered deaths by about 11% while vitamin D2 had no benefit. ULRIKE LEHMANN ET AL., Bioavailability of Vitamin D2 and D3 in Healthy Volunteers, a Randomized Placebo-Controlled Trial, in THE JOURNAL OF CLINICAL ENDOCRINOLOGY AND METABOLISM 4339-45 (2013); RAJIV CHOWDHURY ET AL., Vitamin D and Risk of Cause Specific Death: Systematic Review and Meta-analysis of Observational Cohort and Randomised Intervention Studies, in THE BRITISH MEDICAL JOURNAL 1903 (2014).

Other Vitamins and Minerals

Non-limiting examples of vitamins that may be included in the nutritional supplement disclosed herein include biotin, vitamin A, vitamin C, vitamin E and vitamin K.

Non-limiting examples of minerals which may be included in the nutritional supplement are calcium, magnesium, zinc, iodine, iron, copper, selenium, chromium, phosphorous, chromium, molybdenum and fluoride.

L-Theanine

L-Theanine, is an amino acid and is thought to have a calming effect. In combination with caffeine, it may provide a relaxed alertness as compared to the anxiety and jitteriness of caffeine alone. See SIMON P. KELLY ET AL., L-Theanine and Caffeine in Combination Affect Human Cognition as Evidence By Oscillatory Alpha-Band Activity and Attention Task Performance, in THE JOURNAL OF NUTRITION 1572-1577 (2008). The nutritional supplement preferably includes 100 mg of L-Theanine per serving.

Matrix, Edible Protective Coating and Masking Agents

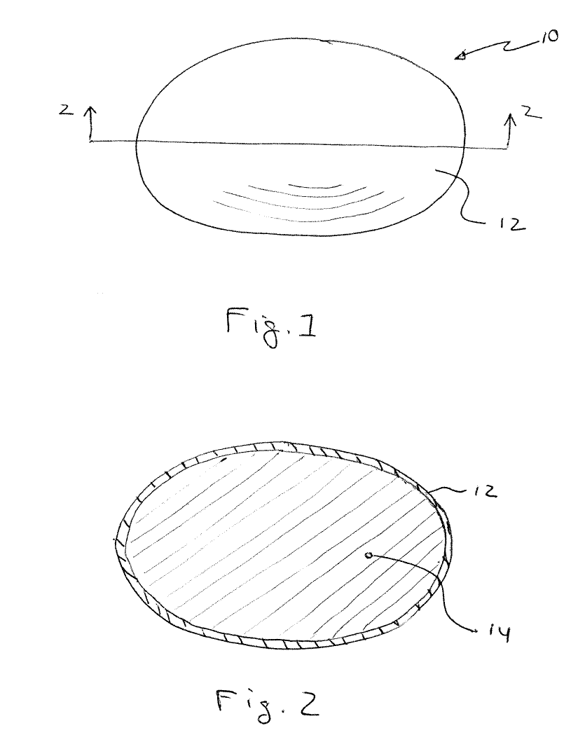

FIG. 1 is perspective view of the nutritional supplement and FIG. 2 is a cross-section of the nutritional supplement at sectional lines 2-2 in FIG. 1. As shown in FIGS. 1 and 2, the nutritional supplement 10 is enveloped by an edible protective coating 12. Encased within the edible protective coating 12 is a fatty matrix 14. The fatty matrix 14 contains the active ingredients—caffeine, vitamins and minerals.

The fatty matrix can be comprised of dark chocolate and the edible protective coating enveloping the fatty matrix can be a sugar shell. The dark chocolate matrix includes a chocolate base in a fill material. The chocolate base comprises known raw materials used in manufacturing of chocolate such as cocoa powder, bitter chocolate and cocoa butter and combinations thereof. The chocolate base further acts as a masking agent to mask the bitterness associated with caffeine or other unpleasant flavors associated with the active ingredients. Further masking agents, such as fruit or other flavors and, in particular, mint and berry flavors, can be included in the matrix to mask the bitterness associated with the caffeine, vitamins and minerals. The matrix preferably includes sugar, chocolate liquor, cocoa butter, soy lecithin (an emulsifier) and vanilla and is standardized to a desired texture, color and flavor. The resulting matrix is a chewy solid or semi-solid when at room temperature such that it can be easily chewed without the need for water.

The edible protective coating can be a soft capsule, a soft gel, a glaze, or a hard shell. One form of a glaze is a chocolate glaze as is known in the art. One form of hard shell is a sugar shell as is known in the confectionery art. The edible protective coating is continuous and preferably airtight, enveloping the fatty matrix. In this manner, the protective coating prevents moisture from infiltrating and spoiling the nutritional supplement. The edible protective coating is not limited to surface coverings that are symmetrical or of identical thickness throughout.

The fat in the matrix, which also prevents moisture infiltration, functions as a preservative for the active ingredients preventing the active ingredients from losing their efficacy or spoiling. The protective coating which continuously envelops the fatty matrix, in conjunction with the matrix, further serves to preserve the active ingredients. As a result of the combination of the matrix and the protective coating, the active ingredients and the nutritional supplement has an enhanced shelf-life such that no artificial preservatives are required.

The fat in the matrix also functions to facilitate bioabsorption of the active ingredients by the human subject. See BESS DAWSON-HUGHES ET AL., Dietary Fat Increases Vitamin D-3 Absorption, in JOURNAL OF THE ACADEMY OF NUTRITION AND DIETETICS 225-230 (2014). The fatty matrix further provides for delivery of caffeine, vitamins and mineral to a human ingesting the food product for enhanced bioabsorption. Preferably, the delivery of the caffeine, vitamins and minerals is constant, consistent and uniform. By providing for delivery and improved bioabsorption of caffeine and vitamin D, the fatty matrix enhances the efficacy and effect of the caffeine and vitamin D in a human subject.

Serving Size/Administration

The delivery system is preferably three pieces per serving, three servings per box, up to one box per day. The ingredient amounts set forth above are for one three piece serving.

The term “comprising” as used herein is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

All numbers in this description indicating amounts of material are to be understood as modified by the word “about.” All amounts are by weight, based on the total weight of the relevant product, unless otherwise specified.

Unless stated otherwise or required by context, the terms “fat” and “oil” are used interchangeably herein.

All references provided herein are incorporated by reference in their entireties.

The specific forms of the nutritional supplement herein illustrated and described are intended to be representative only, as certain changes may be made therein without departing from the clear teaching of the disclosure. Accordingly, reference should be made to the appended claims in determining the full scope of the invention.

What is claimed is:

1. A nutritional supplement having extended shelf life, comprising:
a fatty matrix supporting caffeine and vitamin D;
an edible protective coating enveloping the fatty matrix;
the fatty matrix and edible protective coating cooperating to preserve the caffeine without artificial preservatives.

2. The nutritional supplement of claim 1 wherein the fatty matrix further provides for delivery of caffeine and vitamin D to a human being ingesting the food product for enhanced bioabsorption.

3. The nutritional supplement of claim 1 wherein the Vitamin D helps to maintain muscle function and strength and supports mental health.

4. The nutritional supplement of claim 1 wherein the fatty matrices is comprised of dark chocolate.

5. The nutritional supplement of claim 2 wherein the fatty matrix, by providing for delivery and improved bioabsorption of the caffeine and vitamin D, facilitates a combined, enhanced effect of the caffeine and vitamin D in a human subject.

6. The nutritional supplement of claim 1 wherein the edible protective coating is an airtight sugar shell.

7. The nutritional supplement of claim 1 further comprising L-Theanine.

8. The nutritional supplement of claim 1 further comprising various B vitamins including B3 and B9.

9. The nutritional supplement of claim 8 wherein no Glucuronolactone is included in the supplement.

10. A nutritional supplement having extended shelf life and providing enhanced energy, mental clarity and focus for a human being, comprising:
caffeine;
vitamin D;
a dark chocolate matrix supporting the caffeine and vitamin D;
an edible protective coating enveloping the dark chocolate matrix;
the dark chocolate matrix and edible protective coating cooperating to preserve the caffeine without the need for preservatives or artificial ingredients; and
the dark chocolate matrix further providing for delivery of the caffeine and vitamin D to the human being for enhanced bioabsorption and efficacy.

11. A method of administering a nutritional supplement to a human being to provide enhanced energy, mental clarity and focus, comprising:
administering to the human being a nutritional supplement having
caffeine and vitamin D supported in a dark chocolate matrix;
an edible protective coating enveloping the dark chocolate matrix;
the dark chocolate matrix and edible protective coating cooperating to preserve the caffeine without the need for artificial preservatives; and
the dark chocolate matrix further providing for delivery of the caffeine and vitamin D to the human being for enhanced bioabsorption and efficacy.
